(19) United States
(12) Patent Application Publication
(10) Pub. No.: US 2012/0034191 A1
(43) Pub. Date: Feb. 9, 2012

(54) EXTRACELLULAR MATRIX COMPOSITIONS FOR TISSUE REGENERATION

(76) Inventor: Robert G. Matheny, Norcross, GA (US)

(21) Appl. No.: 13/033,053

(22) Filed: Feb. 23, 2011

Related U.S. Application Data
(63) Continuation of application No. 12/371,158, filed on Feb. 13, 2009, now abandoned.
Application No. 12/371,158 is a continuation of application No. 11/747,018, filed on May 10, 2007, now abandoned.

(51) Int. Cl. A61K 35/38; A61P 1/18; A61P 13/12; A61P 11/00; A61P 15/00; A61P 9/00; A61P 1/16

(52) U.S. Cl. 424/93.7; 424/551

(57) ABSTRACT

The invention is compositions of extracellular matrix that comprise mammalian extracellular matrix from two or more tissue sources in a mammal. The invention also includes methods of using these compositions to regenerate tissue or generate new tissue at sites of defects or wounds in mammals.

### CROSS-REFERENCE TO RELATED APPLICATIONS

This application is a continuation of co-pending U.S. patent application Ser. No. 12/371,158, filed Feb. 13, 2009, which is a continuation of U.S. patent application Ser. No. 11/747,018, filed May 10, 2007, now abandoned, which applications are hereby incorporated herein by reference in their entireties.

### FIELD OF THE INVENTION

The invention relates to regeneration of mammalian tissue using novel compositions of mammalian extracellular matrices.

### BACKGROUND OF THE INVENTION

Extracellular matrices from mammals have been isolated from several mammalian tissues and have been used for tissue regenerative purposes. In particular small intestine submucosa (SIS), liver basement membrane (LBM), urinary bladder submucosa (UBS) and stomach submucosa (SS) have been isolated and described. See U.S. Pat. No. 5,554,389, U.S. Pat. No. 4,902,508, and U.S. Pat. No. 5,281,422. Enamel matrices, which are the extracellular matrix around forming teeth, are described in U.S. Pat. No. 7,033,611. Extracellular matrices from these tissues have been isolated and described as solid materials (sheets and particulates), and in fluidized or emulsion forms made by reconstituting particulate in a suitable buffer. Presently, extracellular matrix is used for tissue grafting, wound healing, and tissue regeneration purposes. It would be advantageous to the field of tissue engineering to devise improved compositions comprising extracellular matrices for tissue regeneration purposes.

### SUMMARY OF THE INVENTION

The invention is a composition comprising mammalian extracellular matrix from two or more mammalian tissue sources.

The invention is also a method comprising identifying a defect or wound in mammalian tissue, providing a composition comprising mammalian extracellular matrix from two or more tissue sources, contacting the defect with a therapeutically effective amount of the composition and regenerating tissue at the defect or healing the wound thereby.

The invention is further a method of regenerating tissue or generating new tissue at a site in a mammalian body comprising locating a site in need of tissue regeneration or generation of new tissue in a mammalian body, providing a composition comprising liquid or emulsion extracellular matrix from one or more mammalian tissue sources, contacting said site with a therapeutically effective amount of said composition, and dusting said composition at said site with a composition comprising extracellular matrix dry particulate.

### DETAILED DESCRIPTION OF THE INVENTION

The invention is compositions of extracellular matrix that comprise extracellular matrices from two or more mammalian tissue sources. The extracellular matrix can be a liquid, emulsion, gel, paste or particulate. The mammalian tissue sources can be any mammalian tissue. The invention is also a composition comprising a liquid, emulsion, gel, or paste that is applied to a site in a mammal and then dusted or covered with an amount of a dry particulate extracellular matrix. The matrix applied to the site, and the dry particulate can be from the same or different mammalian tissue sources.

Mammalian tissue sources are in general any tissue having an extracellular matrix that can be isolated from the mammal and decellularized. Thus for example, mammalian organs are tissue sources. The tissue sources can be for example any mammalian tissue, including but not limited to the small intestine, large intestine, stomach, lung, liver, kidney, pancreas, placenta, heart, bladder, prostate, tissue surrounding growing enamel, tissue surrounding growing bone, and any fetal tissue from any mammalian organ.

The forms of the extracellular matrices that make up the composition are generally particulates, liquids, gels, pastes or emulsions. Liquid is generally a thin emulsion that is injectable and fluid. A rather thicker form is an emulsion or gel which can also be injected if it is not too thick but which has more body and substance than the liquid form. Pastes or near-solid gels or plugs are more concentrated than liquids or injectable emulsions. Particulate is a powder that is formed from the lyophilized sheet broken up in to fine powder or particulate. Particulate can be used dry as a powder. Particulate can also be reconstituted in a suitable buffer such as saline to become liquid or semi-solid forms of the extracellular matrix.

Extracellular matrix can be obtained from the tissues of mammals by processes such as described in U.S. Pat. No. 5,554,389, U.S. Pat. No. 4,902,508, and U.S. Pat. No. 5,281,422. Enamel matrices are described in U.S. Pat. No. 7,033,611 and US Patent publication 2005/0043216. For example, the urinary bladder submucosa is an extracellular matrix that has the tunica mucosa (which includes the transitional epithelial layer and the tunica propria), a submucosal layer, 3 layers of muscularis, and the adventitia (a loose connective tissue layer). This general configuration is true also for small intestine submucosa (SIS) and stomach submucosa (SS).

Other tissues such as the liver and pancreas have extracellular matrix called basement membrane. Basement membrane generally does not demonstrate the kind of tensile strength found in submucosa. However, other useful properties may be opportunistically employed from the extracellular matrices of such tissues as the liver, pancreas, placenta and lung tissues all of which have either basement membrane for extracellular matrix or interstitial membrane (as with the lung). For example, pancreatic extracellular membrane supports beta islet cells which are critical to pancreatic function. Also, for example, the liver is one tissue known to be able to regenerate itself and therefore special qualities may be present in the liver basement membrane that help facilitate that process. The extracellular matrices surrounding developing tooth enamel and developing bone also have particular advantages over other matrices in that they support the growth and differentiation of the hard tissues of bone and enamel.

Matrices can be used in whole or in part, so that for example, an extracellular matrix can contain just the basement membrane (or transitional epithelial layer) with the subadjacent tunica propria, the tunica submucosa, tunica muscularis, and tunica serosa. The extracellular matrix component of the composition can contain any or all of these layers, and thus could conceivably contain only the basement membrane portion, excluding the submucosa, However, generally, and especially since the submucosa is thought to contain and support the active growth factors and other proteins necessary for in vivo tissue regeneration, the matrix composition from any given source will contain the active extracellular matrix portions that support cell development and differentiation and tissue regeneration. For purposes of this invention the extracellular matrix from any of the mammalian tissue consists of several basically inseparable layers broadly termed extracellular matrix. For example, where it is thought that separating a basement membrane from the submucosa is considered to be very difficult if not impossible because the layers are thin and it is not possible to delaminate them from each other, the extracellular matrix from that particular layer will probably necessarily contain some basement membrane with the submucosa. Conversely, it is not likely that a composition that comprises just the basement membrane can be isolated. The composition of the invention then is to two or more of these extracellular matrices from different tissue sources.

Extracellular matrix can be made into a particulate and fluidized as described in U.S. Pat. No. 5,275,826 to Badylak, U.S. Pat. No. 6,579,538 to Spievack, and U.S. Pat. No. 6,933,326 to Griffey. Fluidized or emulsified compositions (the liquid or semi-solid forms) can be present at a certain concentration, for example at a concentration of extracellular matrix greater than about 0.001 mg/ml. The concentration of these liquid or semi-solid components of the extracellular matrix composition can be in a range from about 0.001 mg/ml to about 200 mg/ml. The concentrations can further be found in more specific ranges such as for example the following set of ranges: about 5 mg/ml to about 150 mg/ml, about 10 mg/ml to about 125 mg/ml, about 25 mg/ml to about 100 mg/ml, about 20 mg/ml to about 75 mg/ml, about 25 mg/ml to about 60 mg/ml, about 30 mg/ml to about 50 mg/ml, and about 35 mg/ml to about 45 mg/ml, and about 40 mg/ml. to about 42 mg/ml. This set of ranges is exemplary and not intended to be exhaustive. It is contemplated that any value within any of these specifically listed ranges is a reasonable and useful value for a concentration of a liquid, emulsion, gel, paste or other liquid or semi-solid component of the composition.

The invention is to a composition comprising extracellular matrices from two or more sources of mammalian extracellular matrix. Thus, for example, the composition can comprise extracellular matrix combinations from such sources as, for example but not limited to, small intestine submucosa, liver basement membrane, stomach submucosa, urinary bladder submucosa, placental basement membrane, pancreatic basement membrane, large intestine submucosa, lung interstitial membrane, respiratory tract submucosa, heart extracellular matrix, dermal matrix, and in general extracellular matrix from any mammalian fetal tissue. Any one of these tissue sources can provide extracellular matrix that can then be manipulated into a designated form (liquid, semi-solid, or solid form), for use in a composition.

The compositions of the invention will generally be mixed liquids, mixed emulsions, mixed gels, mixed of pastes, or mixed solid particulates. All of these compositions are mixtures of extracellular matrices from two or more sources, for example mixtures of powders or particulates from two or more extracellular matrices, mixtures of pastes from two or more extracellular matrices, mixtures of emulsions or gels from two or more extracellular matrices and mixtures of liquids from two or more extracellular matrices. To make the compositions, two or more extracellular matrices are isolated from a donor mammal from a particular tissue source in that animal or animals. So that there is an extracellular matrix from one tissue source from a mammal, and there is another extracellular matrix from a different tissue source from either the same mammal, the same species of mammal, or from an entirely different mammalian animal or an entirely different species of mammal. In any event, the key factor is that the at least two tissue sources from which the mammalian extracellular matrix is derived to form the composition are different tissue sources.

The composition can be made from three mammalian tissue sources, four mammalian tissue sources, five mammalian tissue sources, six mammalian tissue sources, and conceivably up to ten or more tissue sources. These tissue sources can be from the same mammal (for example the same cow, the same pig, the same rodent, the same human, etc.), the same species of mammal (e.g. cow, pig, rodent, human), or different mammalian animals, but the same species, (e.g. cow 1 and cow 2, or pig 1 and pig 2), or different species of mammals (for example liver matrix from a pig, small intestine submucosa from a cow, and urinary bladder submucosa from a dog, all mixed together in the composition).

The composition can also be a gel matrix combined with a particulate matrix, where the gel is applied to a space or cavity and dusted with powder-like particulate to increase the concentration of matrix at the surface of the cavity. The composition can be two or more liquid matrices (from different tissue sources) combined together. The composition can be two or more emulsion matrices (from different tissue sources) combined together. The composition can be two or more particulate matrices (from different tissue sources) combined together. The particulate matrices combined together can be applied to the wound or defect as a particulate or as a rehydrated emulsion, where saline or other suitable buffer is applied to the particulate mixture and that hydrated composition is applied to the wound or defect. The particulate can also be dusted onto a sheet of matrix before or after placement at the site in the individual being treated. The composition can be a liquid mixture of two or more extracellular matrices. With this dusting embodiment, the liquid, gel or emulsion can be from a single mammalian tissue source, and dusted with a particulate extracellular matrix from either the same or a different mammalian tissue source. Accordingly, the emulsion, gel or liquid can be SIS, and the particulate can be SIS, or the emulsion, gel or liquid can be SIS and the particulate can be SS, or LBM, or UBS. The emulsion, gel or liquid can be a mixture of SIS and LBM and the particulate for dusting can be from SS. These examples are not meant to be exhaustive of the possible combinations of elements in the possible compositions.

Concentrations of the liquid or semi-solid compositions (liquids, gels, emulsions, or pastes) are important. For example, the liquid forms can be present in a range of concentrations, from very dilute at about 0.001 mg/ml to greater concentrations of up to about 200 mg/ml. The concentrations can further be found in more specific ranges such as for example the following set of ranges: about 5 mg/ml to about 150 mg/ml, about 10 mg/ml to about 125 mg/ml, about 25 mg/ml to about 100 mg/ml, about 20 mg/ml to about 75 mg/ml, about 25 mg/ml to about 60 mg/ml, about 30 mg/ml to about 50 mg/ml, and about 35 mg/ml to about 45 mg/ml, and about 40 mg/ml. to about 42 mg/ml. This set of ranges is exemplary and not intended to be exhaustive. It is contemplated that any value within any of these specifically listed ranges is a reasonable and useful value for a concentration of a liquid or semi-solid component of the composition.

The emulsion will be more concentrated than the liquid form and will retain a shape which can be useful in applying the matrix composition to certain parts of the body, hence its characterization as a “semi-solid”. The emulsion can be concentrated enough to form shapes like a plug or other configuration suited to the site at which the matrix composition is being applied. Thick emulsion can be painted or otherwise applied at a site as a paste, and dusted with solid particulate on top of the emulsion. The solid particulate can be reconstituted to form the emulsion, or can be applied dry as a particulate powder which can dust a region in the subject being treated

The dry particulate or reconstituted particulate that forms an emulsion of the two matrices can be mixed together in some proportion. For example, 50% of small intestine submucosa can be mixed with 50% of pancreatic basement membrane in a vial. This mixture can then be fluidized by hydrating it in a suitable buffer, for example saline. The hydration can be accomplished to a desired concentration of the extracellular matrix mixture, for example in a range from about 0.001 mg/ml to about 200 mg/ml. The concentrations can further be found in more specific ranges such as for example the following set of ranges: about 5 mg/ml to about 150 mg/ml, about 10 mg/ml to about 125 mg/ml, about 25 mg/ml to about 100 mg/ml, about 20 mg/ml to about 75 mg/ml, about 25 mg/ml to about 60 mg/ml, about 30 mg/ml to about 50 mg/ml, and about 35 mg/ml to about 45 mg/ml, and about 40 mg/ml. to about 42 mg/ml. This set of ranges is exemplary and not intended to be exhaustive. It is contemplated that any value within any of these specifically listed ranges is a reasonable and useful value for a concentration of a liquid or semi-solid component of the composition.

The lower the concentration of extracellular matrix the more liquid the composition will be and the higher the concentration of extracellular matrix the more the composition approaches a gel-like emulsion or semi-solid consistency.

The ratio of the mixtures of the two (or more) extracellular matrices in any given composition from different sources (or the same source) can be unequal. So for example, LBM can be present at 75% and SIS can be present at 25% (e.g. a 3:1 ratio). Any suitable ratio can be used: 1:1, 1:2, 1:3, 1:4, 1:5, and so on. Where 3 or more tissue sources of extracellular matrix are represented in the composition, the same type of balance or imbalance in the amounts of the matrices can occur. For extracellular matrix from 3 sources, each source can be present in a third, 1:1:1, or a disproportionate amount of the particulate can be from one source, say 50% and the other two sources can be present in equal or unequal amounts (relative to each other), so as 25% each, or one can be present as 30% and the other as 20%, so 1:2:3, and 1:1:2 ratios for example.

The two or more matrices can be fluidized (or emulsified) separately and the fluidized or emulsified compositions mixed together. As another alternative, the two or more matrices can be fluidized or emulsified separately, and applied to the defective myocardial tissue separately. In addition, the two or more different matrices can remain in particulate solid form and be mixed together in a vial for application to the site of defect as a solid combination particulate. Also, alternatively, these two or more matrices can be applied to the site of defect at approximately the same time (in the same procedure) but without mixing them together first. Rehydration of the dry particulate acellular tissue matrix mixture can be accomplished just prior to use.

The particulate acellular matrix compositions can be applied to the site of tissue defect by injecting an emulsified composition, spraying, layering, packing, dusting, painting, or other similar types of application of the dry particulate, the liquid composition, or the semi-solid compositions.

The invention is compositions that are mixtures of extracellular matrix from two or more tissue sources. Each composition therefore has at least a mixture of two extracellular matrices (e.g. SIS+LBM; SS+SIS, pancreatic basement membrane+SIS; etc.). In some cases an emulsion can be covered with powder after placement in the body, therefore providing a composition having extracellular matrix in two forms, even if the emulsion is SIS and the powder is also SIS. This latter example would be a way of further concentrating the composition that is being used, so that the exterior surface of the composition has a greater concentration of extracellular matrix than the interior portion of the composition.

The composition comprising mammalian extracellular matrix from different tissue sources can further include one or more additional components to aid in some aspect of the tissue regenerative process or the wound healing or generation of new tissue, however the biological activity is characterized. The additional component can be any component that some how serves the composition and its purpose in the mammalian body. Thus, the additional component can help to regenerate tissue, heal a wound, better recruit stem cells, manipulate the immune environment in a beneficial way, therapeutically treat the local environment, or otherwise contribute to some aspect of the process for which the composition is being used.

Thus, the additional component can be a cell, a protein or a drug. The cell can be a stem cell, such as, for example a of human embryonic stem cell, a fetal cardiomyocyte, a myofibroblast, a mesenchymal stem cell, an autotransplanted expanded cardiomyocyte, an adipocyte, a totipotent cell, a pluripotent cell, a blood stem cell, a myoblast, an adult stem cell, a bone marrow cell, a mesenchymal cell, an embryonic stem cell, a parenchymal cell, an epithelial cell, an endothelial cell, a mesothelial cell, a fibroblast, a myofibroblast, an osteoblast, a chondrocyte, an exogenous cell, an endogenous cell, a stem cell, a hematopoetic stem cell, a pluripotent stem cell, a bone marrow-derived progenitor cell, a progenitor cell, a myocardial cell, a skeletal cell, a fetal cell, an embryonic cell, an undifferentiated cell, a multi-potent progenitor cell, a unipotent progenitor cell, a monocyte, a cardiomyocyte, a cardiac myoblast, a skeletal myoblast, a macrophage, a capillary endothelial cell, a xenogenic cell, an allogenic cell, an adult stem cell, and a post-natal stem cell. This list is not intended to be exhaustive.

The protein can be for example a growth factor, or any other type or protein that might stimulate some part of the tissue regenerative, wound healing, or new tissue generating process. The protein can be a collagen, a proteoglycan, a glycosaminoglycan (GAG) chain, a glycoprotein, a growth factor, a cytokine, a cell-surface associated protein, a cell adhesion molecule (CAM), an angiogenic growth factor, an endothelial ligand, a matrikine, a matrix metalloprotease, a cadherin, an immunoglobin, a fibril collagen, a non-fibrillar collagen, a basement membrane collagen, a multiplexin, a small-leucine rich proteoglycan, decorin, biglycan, a fibromodulin, keratocan, lumican, epiphycan, a heparan sulfate proteoglycan, perlecan, agrin, testican, syndecan, glypican, serglycin, selectin, a lectican, aggrecan, versican, nuerocan, brevican, cytoplasmic domain-44 (CD-44), macrophage stimulating factor, amyloid precursor protein, heparin, chondroitin sulfate B (dermatan sulfate), chondroitin sulfate A, heparan sulfate, hyaluronic acid, fibronectin (Fn), tenascin, elastin, fibrillin, laminin, nidogen/entactin, fibulin I, fibulin II, integrin, a transmembrane molecule, platelet derived growth factor (PDGF), epidermal growth factor (EGF), transforming growth factor alpha (TGF-alpha), transforming growth factor beta (TGF-beta), fibroblast growth factor-2 (FGF-2) (also called basic fibroblast growth factor (bFGF)), thrombospondin, osteopontin, angiotensin converting enzyme (ACE), or a vascular epithelial growth factor (VEGF). This list is not intended to be exhaustive.

The additional component can also be a drug, such as an agent that has therapeutic properties. The drug can be bioactive and play some role in the process of tissue regeneration or act as an antibiotic, antiviral, or other active therapeutic agent serving a purpose in the composition as a whole. The drug can be a small molecule, or any other agent having therapeutic properties.

The invention contemplates using the compositions of extracellular matrices from two or more sources for contacting a defect or wound in mammalian tissue. The compositions can also be used to augment regions of tissue deficiency in the human subject being treated. The defect or wound can be a cut, disease, wound, burn, scar, necrosis, or other abnormality that would be beneficial to the patient to treat. Regenerating tissue at the defect can be one response elicited from the step of placing the extracellular matrix composition in contact with the defect. If the defect is a wound in need of healing, wound healing may be another response that occurs as a result of placing the extracellular matrix at the wound site. Further, the biological activity might be characterized as generating new tissue at a site. In general any term that identifies that the tissue could benefit from a healing or tissue regeneration fits within the scope of the use for the composition. Thus regenerating tissue, healing a wound, or generating new tissue are three such phrases to describe the biological process that is hoped for, but the not the only phrases that can be used to describe the effects achieved when the composition is placed in the mammal at a site of defect or damage in tissue.

The compositions can be used to fill a tumor excision site after excision of the tumor. The composition will promote wound healing at the site and tissue regeneration of the lost tissue.

The compositions can be used to reconstruct or augment breast tissue. A mixture of emulsions can be placed in the breast, or a mixture or particulates can be placed in the breast, and new breast tissue regenerates. If more emulsion or particulate is placed in the breast, than was previously there in the native mammalian tissue, then augmentation of the breast or enhancement of the breast size results after the extracellular matrix composition forms new tissue in the breast. Other tissues in the body can also be augmented where deemed necessary.

Therapeutically effective amount is a term meant to capture the idea that you need to apply enough of the composition in sufficient strength so that the composition can have a positive effect on the tissue that is being treated in the subject. The amount may therefore apply to a quantity of matrix, or a volume or weight of powder, or a concentration of liquid or emulsion. That the amount is therapeutically effective is determined by the composition's ability to have a regenerative or wound healing effect at the site where the composition contacts the tissue. A therapeutically effective amount is determinable by routine testing in patients with wounds or defects. In general a minimal therapeutically effective amount would be considered sufficient composition to contact amply all of the wound or defect in the tissue, or to fill the tumor excision space, or otherwise act at the site to regenerate tissue, heal the wound at the site, or generate new tissue.

Regenerating tissue is the ability to make tissue regrow, an organ regrow itself, and for tissue to reform without scarring. Healing a wound is the ability of the tissue to heal without scarring. Forming new tissue may be the result of tissue regeneration, and as such could be characterized as a process of generating new tissue. Reconstructing a breast involves both regenerating new tissue in the tissue space and healing a wound in the breast caused by surgical removal of tissue such as a tumor, or by the placement in the breast of an implant. The implant can be made of extracellular matrix, and thus both the space filing agent and the wound healing agent are one in the same for the purposes of breast reconstruction and augmentation. A sufficient amount of the composition needs to be applied to the breast in order to properly reconstruct the tissue, and augment the breast size. After a determination of the target size for the breast, a sufficient amount of the composition of extracellular matrix emulsion or particulate will then be that amount that will allow the breast to regenerate tissue to the predetermined target size.

### EXAMPLES

### A. Example 1

Several compositions are made to test in animals for tissue regeneration and wound healing purposes: an emulsion composition of the following ratios of SIS to LBM: 3:1, 1:1, and 1:3. The animals tested are observed for comparative purposes to see which of the three ratios perform the best for the purposes identified. The same ratios are tested for the following mixtures of extracellular matrices in liquid, emulsion, paste, and particulate forms: SS+UBS, SIS+pancreatic basement membrane, SIS+placental extracellular matrix, SIS+fetal heart extracellular matrix. The animals are observed for comparative purposes to see both which combinations of matrices work best, and which ratios of these combinations. The compositions are placed in defects in selected tissues including heart, pancreas, liver, lung, reproductive tissues, and kidney.

All references cited are incorporated in their entirety. Although the foregoing invention has been described in detail for purposes of clarity of understanding, it will be obvious that certain modifications may be practiced within the scope of the appended claims.

What is claimed is:

1. A composition comprising mammalian extracellular matrix from small intestine submucosa (SIS) and a mammalian extracellular matrix selected from one or more tissue sources selected from the group consisting of stomach submucosa (SS) and urinary bladder submucosa (UBS).

2. The composition of claim 1, wherein the composition comprises mammalian extracellular matrix from SIS and SS.

3. The composition of claim 1, wherein the composition comprises mammalian extracellular matrix from SIS and UBS.

4. The composition of claim 1, further comprising a cell.

5. The composition of claim 1, further comprising a protein.

6. The composition of claim 1, further comprising a drug.

7. A method comprising
(a) identifying a site of defect or wound in mammalian tissue,
(b) providing a composition comprising mammalian extracellular matrix from small intestine submucosa (SIS) and a mammalian extracellular matrix selected from one or more tissue sources selected from the group consisting of stomach submucosa (SS) and urinary bladder submucosa (UBS),
(c) contacting said site with a therapeutically effective amount of said composition, and
(d) regenerating tissue or generating new tissue at said site thereby.
